(19) United States
(12) Patent Application Publication
(10) Pub. No.: US 2009/0028957 A1
(43) Pub. Date: Jan. 29, 2009

(54) Implantable Tissue-Reactive Biomaterial Compositions and Systems, and Methods of Us Thereof

(76) Inventor: George Y. Daniloff, Mountain View, CA (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/083,646

(22) PCT Filed: Apr. 19, 2006
(86) PCT No.: PCT/US2006/014832

§ 371 Date: Apr. 14, 2008

Related U.S. Application Data
(60) Provisional application No. 60/594,559, filed on Apr. 19, 2005.
(60) Provisional application No. 60/594,560, filed on Apr. 19, 2005.
(60) Provisional application No. 60/594,561, filed on Apr. 19, 2005.

(51) Int. Cl. A61K 9/14; A61M 31/00

(52) U.S. Cl. 424/501; 604/95.01

(57) ABSTRACT

The invention pertains to biomaterial compositions and implantable systems for application to a tissue site in a living mammalian body. The compositions and systems react with tissue surfaces and components present in physiological fluids at the region of implantation, obviating the need to remove such fluids and improving the adhesion and resorption rate of the resulting bioimplant. Delivery devices and methods of use are also provided.

### CROSS-REFERENCE TO RELATED APPLICATIONS

This application claims priority under 35 U.S.C. § 119 to the following Provisional U.S. Patent Application Ser. No. 60/594,559, filed Apr. 19, 2005; Provisional U.S. Patent Application Ser. No. 60/594,560, filed Apr. 19, 2005; and Provisional U.S. Patent Application Ser. No. 60/594,561, also filed Apr. 19, 2005. The disclosures of the aforementioned applications are incorporated by reference herein.

### TECHNICAL FIELD

This invention relates generally to biomaterials used for medical purposes, and more particular relates to biomaterial compositions and implantable systems for introduction into a living mammalian body at a selected tissue site. The invention finds utility in the field of medicine, particularly in surgery, wound healing, and drug delivery.

### BACKGROUND

Many challenges and clinical needs in surgery can be successfully addressed with the use of biocompatible, non-toxic, non-immunogenic, and optionally resorbable biomaterials. For instance, such biomaterials are find utility in the control of bleeding and fluid leakage, in the prevention of postsurgical tissue adhesions, in implant fixation and immobilization, in tissue repair and regeneration, in facilitating local drug delivery, and in effecting sutureless wound closure. Several in vivo gelling formulations of biomaterials have been commercially developed. Most of these biomaterials are formed in vivo from reactive water-soluble polymers or from water-soluble blood derived products. There remains, however, an ongoing substantial need for single-component, simple-to-use products that do not require reagent preparation, dissolution prior to use, and/or mixing of reactive reagents prior to delivery to the tissue site.

In vivo surgical wound surfaces are always covered with a layer of physiological fluid, and often they are actively bleeding or leaking fluid and gases. The fluids present at the wound or surgical site may include any of a number of physiological fluids such as blood, lymph, plasma, wound exudate, seroma fluid, peritoneal fluid, cerebrospinal fluid, and bone marrow. Several commercially available products use two-component sealant systems that gel at the tissue site, following admixture of the two reactive components immediately prior to application. The reactive components are natural or synthetic polymers that crosslink when combined under appropriate conditions. It is desirable that the formed gel or sealant be covalently bound to the tissue surface; however, the presence of physiological fluids or excess of moisture on the surface of the tissue often hinders such covalent binding. In fact, currently available products often require removal of tissue fluids prior to use, e.g., via blotting or rinsing.

A preferred biomaterial would, therefore, covalently bind to a tissue surface without need for removing blood or other physiological fluids prior to application. An ideal biomaterial would make use of physiological fluid present instead of requiring its removal. In one embodiment, the invention is directed to such a biomaterial, and in a preferred embodiment the invention provides a biomaterial composition that reacts with physiological fluid present at the tissue site. This feature of the invention provides a substantial improvement in the field, not only obviating the need for fluid removal prior to application of a biomaterial to a tissue site, but also enhancing the binding of the biomaterial to the tissue surface.

### SUMMARY OF THE INVENTION

Accordingly, the invention is directed to the aforementioned needs, providing, in one embodiment, a biomaterial composition that is advantageous relative to previously known or described biomaterials requiring removal of physiological fluid prior to application. In a first aspect of the invention, a biomaterial composition is provided that comprises particles that are in turn comprised of a biocompatible polymer, wherein the particles have tissue-reactive groups that react with endogenous nucleophilic groups present at a tissue site in a mammalian body to form new covalent bonds, ionic bonds, or both covalent bonds and ionic bonds. The particles are in a particle population having a median diameter in the range of about 1 nm to about 3 mm, preferably about 1 μm to about 100 μm, more preferably about 25 μm to about 75 μm.

In general, the biomaterial composition comprises a resorption rate-controlling component that controls the rate at which the composition is resorbed in the body. This is ensured by selecting a component with an aqueous solubility such that the composition is resorbed in not less than 3 hours following introduction into a living mammalian body at a selected tissue site. The tissue-reactive groups are functional groups or other molecular segments that react with electrophilic or nucleophilic moieties present at the tissue site, e.g., amino and sulfhydryl groups on peptides, proteins, cell surfaces, and extracellular matrix components, to form covalent bonds. Generally, and as will be appreciated by those skilled in the art, the moieties at the tissue site are nucleophilic.

The biomaterial may be incorporated into or serve as an implant, i.e., an implantable, generally resorbable composition, or it may be incorporated into a resorbable or non-resorbable medical device. Examples of suitable devices include, without limitation, hernia repair meshes, nasal tubes, ear tubes, pins, clips, vascular prostheses, anastomotic stents, nerve anastomosis tubes, bronchial anastomosis stents and tubes, intestinal anastomosis stents and tubes, esophageal anastomosis stents, extraesophageal tubes, ureter stents and tubes, intravascular stents, extravascular stents, vascular bypass prostheses and shunts, embolic particles, spheres and beads, film, tape, rods, fibers, sutures, drainage tubes, catheters, hip implants, bone implants, cartilage implants, and spinal disc implants.

In a second aspect of the invention, a biomaterial composition is provided that comprises a population of porous particles having a median diameter in the range of about 1 nm to about 3 mm, wherein the particles have tissue-reactive groups that, as described above with respect to the first aspect of the invention, react with endogenous nucleophilic groups present at a tissue site in a mammalian body to form new covalent bonds, ionic bonds, or both covalent bonds and ionic bonds. The composition also includes a biocompatible polymer associated with the particles but not covalently bound thereto. Here as well, the composition preferably includes a resorption rate-controlling component that controls the rate at which the composition is resorbed in the body, i.e., a component with an aqueous solubility such that the composition is resorbed in not less than 3 hours following introduction into a living mammalian body at a selected tissue site.

In a third aspect of the invention, a biocompatible, implantable system is provided for implantation into a mammalian body at a tissue site to form a “bioimplant.” The implantable system comprises: (a) a biomaterial composition having tissue-reactive groups selected from electrophilic groups and nucleophilic groups that react with endogenous groups present at a tissue site in a mammalian body to form new covalent bonds, ionic bonds, or both covalent bonds and ionic bonds, wherein, if the biomaterial composition has both electrophilic groups and nucleophilic groups, then under predetermined conditions (e.g., with respect to pH, heat, light, or the presence of a particular reagent) either the electrophilic groups or the nucleophilic groups, but not both, react with the endogenous groups; and (b) a resorption rate-controlling component as described above, having an aqueous solubility selected such that the composition is resorbed after introduction into a living mammalian body over a time period of not less than 3 hours.

In a fourth aspect of the invention, a biocompatible, implantable system is provided for implantation into a mammalian body at a tissue site to form a bioimplant, the composition comprising: (a) porous particles having a median diameter in the range of about 1 μm to about 100 μm and comprising a material selected such that the bioimplant is resorbed over a time period of not less than 3 hours; and (b) impregnated therein, a biocompatible polymer having tissue-reactive electrophilic groups that react with endogenous nucleophilic groups present at the tissue site to form new covalent bonds, ionic bonds, or both covalent bonds and ionic bonds.

The aforementioned systems are generally stored in substantially anhydrous form. The systems can also include buffer components to ensure a particular pH in the region where the system is implanted. A particular pH, depending on the type of tissue-reactive group(s) present, may be useful in controlling the initialization and/or extent of reaction between the tissue-reactive groups and endogenous groups present at the tissue site. In other embodiments, the systems may include an effective amount of an active agent, a nonpolymeric material capable of polymerization upon contact with the tissue surface, a polymerization initiator, a visualization component, and/or at least one additional component selected from living cells, stem cells, autologous cells, cell fragments, non-living cells, viruses, plasmids, prions, and bacteria.

In a fifth aspect, a bioimplant is provided that results from implantation into a living mammalian body at a selected tissue site of a biomaterial composition or implantable system of the invention. The “bioimplant” (i.e., an implant that forms in situ following application of the biomaterial to a tissue surface) comprises the biocompatible composition or implantable system covalently bound to a surface of a tissue in a living mammalian body and to components of endogenous physiological fluids present in the region of the tissue. Accordingly, the “bioimplant” is composed of bound or crosslinked components of physiological fluids and said particles. The bioimplant may be resorbable or non-resorbable, polymeric or non-polymeric, swellable or non-swellable, flexible or rigid, porous or non-porous, and hydrophilic or hydrophobic.

In a sixth aspect of the invention, a biomaterial composition is applied to a tissue site within a living mammalian body, but rather than forming a bioimplant per se, diffuses from the site of application, e.g., from a medical device, delivery system, or other applicator, to crosslink surrounding tissue components. In this case, a sutureless device may be used that is comprised of a biocompatible tissue crosslinker that is not covalently attached to the device surface. The crosslinker is able to diffuse from the device and crosslink surrounding tissue components. For example, a tissue penetrating arrow-shaped wound closure device can be impregnated with a crosslinker that can diffuse out and crosslink the tissue and optionally the chemical groups on the device once it is inserted into the tissue. In a seventh aspect of the invention, a device is provided for implanting a biocompatible composition or implantable system of the invention into a living mammalian body at a predetermined tissue site. The device houses the composition or system and further includes a means for introducing the composition into the body at the predetermined tissue site, generally to form a bioimplant in the region of the implantation site. In one embodiment, a propellant driven delivery system is provided which comprises the implantable composition or system, a propellant, and a device that contains the composition and the propellant and includes a means for ejecting the composition in aerosolized form. The propellant may be housed in a first compartment of the device, with the composition housed in a second compartment of the device, such that upon ejection through a nozzle, the composition and propellant are combined upon aerosolization.

In an eighth aspect of the invention, methods are provided for using the aforementioned biomaterial composition, implantable system, and implantation device. For instance, the invention is useful in the surgical repair of living tissue at a tissue site wherein joinder of adjacent but separate (e.g., ruptured) regions of tissue exist. Surgical repair, as will be appreciated, includes wound closure, surgical sealing, and the like. Another method in which the compositions, systems, and devices of the invention are useful is for the delivery of an active agent to a tissue site in the body. The active agent is typically intended for local delivery in the region of the tissue site where the composition or system is implanted.

In a ninth aspect of the invention, a device for wound closure or surgical repair is provided that has a size and shape suitable for application to a selected tissue site in need of closure or repair. The device is comprised of: (a) a biomaterial composition having tissue-reactive groups selected from electrophilic groups and nucleophilic groups that react with endogenous groups present at a tissue site in a mammalian body to form new covalent bonds, ionic bonds, or both covalent bonds and ionic bonds, wherein, if the biomaterial composition has both electrophilic groups and nucleophilic groups, then under predetermined conditions (as explained supra) either the electrophilic groups or the nucleophilic groups, but not both, react with the endogenous groups; and (b) a resorption rate-controlling component having an aqueous solubility selected such that the composition is resorbed after introduction into a living mammalian body over a time period of not less than 3 hours.

The mixing of the implant with physiological fluids may be aided by the use of surfactants, foaming agents, outgassing compositions and by externally induced means such as flowing gaseous or liquid propellant.

In some embodiments the invention makes use of the swelling properties of many hydrophilic materials. Applying dry or partially hydrated polymeric materials to the tissue site leads to absorption of tissue fluids by the material and hydrostatic swelling of the implanted polymer. Swelling of implanted materials can often be beneficial as it applies pressure on local tissues and can have a sealing and hemostatic effect. In some other medical conditions swelling of implanted materials is unacceptable, such as in neurosurgery, e.g., spinal surgery. In such clinical indications the non-swelling medical implants are used instead. In one embodiment of this invention the swelling characteristics of a sutureless closure device is used to anchor the tissue-inserted portion of the device inside the tissue. Reaction of reactive groups, which may be co-delivered with said device, further help to reinforce and secure the swelling device to the tissue.

A number of biomaterial compositions have recently been developed for forming implantable biomaterials in vivo. Such compositions include the in vivo polymerization or crosslinking of hydrophilic polymers having multiple reactive groups. Typically, two liquid components containing reactive groups, for example one component having electrophilic groups and other components having nucleophilic groups, are mixed and react at the tissue site forming a hydrogel-like material. Cyanoacrylates and other acrylates are used to form a polymeric implant by polymerizing monomer or prepolymer liquid components into a solid implant. Commercially available products, such as CoSeal (Baxter), FocalSeal (Genzyme Biosurgery), DuraSeal (Confluent Surgical) and others, make use of liquid reagents forming an implant in vivo and are used as tissue sealants, hemostats and adhesives. While implants formed from these products can be bound to the tissue surface as a result of reactive groups that are used to form the implant also reacting with nucleophilic groups on the tissue surface, the strength of the implant-tissue interface is limited by the structure of the surface layer of the tissue. The strength of the implant-tissue interface is particularly important for surgical glues and adhesives. The tissue reactive groups of some surgical glues and adhesives typically form a covalent bond with proteins on the surface of tissue cells. Many surface cells with a crosslinked cell surface die or shed the crosslinked proteins from the surface. As a result the bond between the implant and the tissue surface becomes weaker over time.

Sutures and staples have been successfully used for surgical tissue approximation, repair, and wound closure. In contrast to surface reactive glues and adhesives that rely on the strength of a thin tissue-biomaterial interface, sutures and staples rely on fasteners which penetrate through living tissue to bear and anchor the mechanical load of the surgical repair.

In a further aspect of the invention, then, devices for sutureless wound closure or other surgical repair are provided, which combine the advantages of tissue reactive surgical adhesives with the advantages of sutures and staples. The device is shaped to penetrate and anchor into the tissue, and it contains tissue reactive groups or polymer forming compositions capable of immobilizing the device inserted into the tissue and strengthening the wound closure site. The devices that can be made to have sutureless tissue immobilization ability include, but are not limited to hernia repair meshes, nasal tubes, ear tubes, pins, clips, vascular prostheses, anastomotic stents, nerve anastomosis tubes, bronchial anastomosis stents and tubes, intestinal anastomosis stents and tubes, esophageal anastomosis stents and extraesophageal tubes, ureter stents and tubes, intravascular stents, extravascular stents, vascular bypass prosthesis and shunts, embolic particles, spheres and beads, film, tape, rods, fibers, sutures, drainage tubes, catheters, hip implants, bone implants, cartilage implants, spinal disc implants, eye implants, ear implants, heart implants, etc.

### DETAILED DESCRIPTION OF THE PREFERRED EMBODIMENTS

Unless otherwise indicated with respect to a particular embodiment, the invention is not limited to specific compositions, components, active agents, delivery systems or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a biocompatible polymer” includes a single such polymer as well as two or more such polymers that may be the same or different, reference to “a biomaterial” encompasses a combination or mixture of different biomaterials as well as a single biomaterial, and the like.

In this specification and in the claims that follow, reference will be made to a number of terms, which shall be defined to have the following meanings:

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. If not otherwise indicated, the term “alkyl” includes linear, branched, cyclic, unsubstituted, substituted, and/or heteroatom-containing alkyl.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, preferably 2 to 12 carbon atoms. If not otherwise indicated, the term “alkenyl” includes linear, branched, cyclic, unsubstituted, substituted, and/or heteroatom-containing alkenyl.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. If not otherwise indicated, the term “aryl” includes unsubstituted, substituted, and/or heteroatom-containing aromatic substituents.

The term “alkaryl” refers to an aryl group with an alkyl substituent, and the term “aralkyl” refers to an alkyl group with an aryl substituent, wherein “aryl” and “alkyl” are as defined above.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene et al., Protective Groups in Organic Synthesis (New York: Wiley, 1991).

When a functional group is termed “activated,” this refers to a modification of an existing functional group to generate or introduce a new reactive functional group, wherein the new reactive functional group is capable of undergoing reaction with another group to form a covalent bond. For example, a component containing carboxylic acid (—COOH) groups can be activated by reaction with N-hydroxy-succinimide or N-hydroxysulfosuccinimide using known procedures, to form an activated carboxylate (which is a reactive electrophilic group), i.e., an N-hydroxysuccinimide ester or an N-hydroxysulfosuccinimide ester, respectively. In another example, carboxylic acid groups can be activated by reaction with an acyl halide such as acyl chloride to provide an activated electrophilic group in the form of an anhydride.

The term “implant” as used herein refers to any composition or object placed surgically or otherwise in contact with a human or animal body. Such implants can have a diagnostic, therapeutic, or aesthetic function, or can be used as identification or information storage or processing devices. Implants can be attached on the outside surfaces of the body such as skin, oral mucosa, teeth, nails, eye, and ear-nose-throat passages, or can be placed inside of the pulmonary system, digestive system, urinary system, intestinal tract, reproductive system, or vascular system, or surgically placed subcutaneously, intramuscularly, intaperitoneally, or in any other location in the body. Generally, the term “implant” as used herein refers to a composition or system rather than a device per se.

The term “bioimplant” refers to a composition of matter formed at a tissue site following implantation of a composition or system as described herein, the composition or system having multiple reactive groups. Placing such a composition or system onto or into the tissue allows the implant to mix with the physiological fluids present at the tissue site and react with the tissue surface. The formed bioimplant is thus comprised of the composition or system and is bound and/or crosslinked by virtue of the reactive groups present on components of physiological fluids and may be directly or indirectly covalently bound to the tissue surface.

The term “crosslinked” herein refers to a composition containing intermolecular crosslinks and, optionally, intramolecular crosslinks as well, arising from the formation of covalent bonds. Covalent bonding may be direct, in which case an atom in one component is directly bound to an atom in the other component, or it may be indirect, through a linking group.

The term “water insoluble” herein means difficult or incapable of being dissolved in water, or in physiological fluids forming a true molecular solution upon delivery at the tissue site. The term “water insoluble” refers to insolubility over the several hours or days and does not refer to inability to be resorbed or hydrolyzed at the tissue site over longer periods of time. For example, hydrogel particles may have high water content, however, they are deemed water insoluble as they are not dissolved at the tissue site, continue to maintain their shape and have certain material characteristics (i.e. strain and elastic modulus) upon delivery to the tissue site. If water insoluble compositions contains hydrolysable or resorbable chemical bonds it will eventually be dissolved at the tissue site, but over a much longer time them required to dissolve a water-soluble composition yielding true molecular solution. Another example of a “water insoluble” system is a colloidal solution in which finely divided particles of approximately 10 to 10,000 angstroms in size, are dispersed within a continuous medium in a manner that prevents them from being filtered easily or settled rapidly. Other examples of water insoluble systems include suspensions, dispersions, slurries, pastes, emulsions, and water insoluble solids. Generally, when the “water-insoluble” resorption rate-controlling component is in a composition or system of the invention that has been implanted in the body, less than 5 wt. %, preferably less than 1 wt. % of the component will dissolve within an hour of implantation and contact with aqueous physiological media

The term “nucleophilic” refers to a functional group that is electron rich, has an unshared pair of electrons acting as a reactive site, and reacts with a positively charged or electron-deficient site, generally present on another molecule. The term “nucleophile” refers to a compound having a nucleophilic site.

The term “electrophilic” refers to a functional group that is susceptible to nucleophilic attack, i.e., susceptible to reaction with an incoming nucleophilic group. Electrophilic groups herein are typically electron-deficient. The term “electrophile” refers to a compound having an electrophilic site. General examples of electrophilic reactive groups include (1) alkenyloxycarbonyl groups, i.e., carboxylic acid esters, and “activated” esters; (2) halocarbonyl groups such as acid chloride groups (—CO—Cl); (3) anhydrides (—(CO)—O—(CO)—R, where R is substituted or unsubstituted alkyl, aryl, alkaryl, etc.); (4) acyl groups (ketones) and formyl groups (aldehydes), including α,β-unsaturated ketones and aldehydes (e.g., —CH═CH—CH═O and —CH═CH—C(CH₃)═O); (5) halides, particularly chloro substituents; (6) isocyano groups (—N═C═O); (7) isothiocyano groups (—N═C═S); (8) epoxides; (9) activated hydroxyl groups (e.g., activated with conventional activating agents such as carbonyldiimidazole or sulfonyl chloride); and (10) alkenyl groups, including conjugated olefins, such as ethenesulfonyl (—SO₂ CH═CH₂) and analogous functional groups, including acrylate (—CO₂—C═CH₂), methacrylate (—CO₂—C(CH₃)═CH₂)), ethyl acrylate (—CO₂—C(CH₂CH₃)═CH₂), and ethyleneimino (—CH═CH—C═NH).

Further examples of electrophilic tissue reactive groups include, without limitation: mixed anhydrides such as PEG-glutaryl-acetyl-anhydride; PEG-glutaryl-isovaleryl-anhydride; PEG-glutaryl-pivalyl-anhydride; ester derivatives of p-nitrophenol, p-nitrothiophenol, and pentafluorophenol; esters of substituted hydroxylamines such as those of N-hydroxy-phthalimide, N-hydroxy-succinimide, and N-hydroxy-glutarimide; esters of 1-hydroxybenzotriazole, 3-hydroxy-3,4-dihydrobenzotriazine-4-one and 3-hydroxy-3,4-dihydro-quinazoline-4-one; derivatives of carbonylimidazole; and isocyanates. With these compounds auxiliary reagents can also be used to facilitate bond formation. For example 1-ethyl-3-(3-dimethylaminopropyl)]carbodiimide can be used to facilitate coupling of carboxyl groups (i.e., glutarate and succinate) with sulfhydryl groups.

In addition to the sulfhydryl reactive compounds that form thioester linkages, various other compounds can be utilized that form other types of linkages. For example, compounds that contain methyl imidate derivatives form imido-thioester linkages with sulfhydryl groups. Alternatively, sulfhydryl reactive groups can be employed to form disulfide bonds with sulfhydryl groups, such as ortho pyridyl disulfide, 3-nitro-2-pyridenesulfenyl, 2-nitro-5-thiocyanobenzoic acid, 5,5′-dithio-bis(2-nitrobenzoic acid), derivatives of methane-hyposulfite, and 2,4-dinitrophenyl cysteinyl disulfides. In such instances auxiliary reagents such as hydrogen peroxide or the di-tert-butyl ester of azodicarboxylic acid can be used to facilitate disulfide bond formation.

The terms “hydrophilic” and “hydrophobic” are generally defined in terms of a partition coefficient P, which is the ratio of the equilibrium concentration of a compound in an organic phase to that in an aqueous phase. A hydrophilic compound has a log P value of less than 1.0, typically of less than about 0.5, where P is the partition coefficient of the compound between octanol and water, while hydrophobic compounds will generally have a log P greater than about 3.0, typically greater than about 5.0.

The term “polymer” is used to refer to molecules composed of repeating monomer units, including homopolymers, block copolymers, random copolymers, and graft copolymers. All molecular weights herein are weight average molecular weights.

The term “prepolymer” refers to monomers, oligomers and polymers that can be further used to create a larger molecular weight polymer by crosslinking, polymerizing or otherwise covalently linking prepolymers.

The term “small molecular weight crosslinker” refers to multi reactive group containing molecules with molecular weights between approximately 100 and 3,000 Daltons. For example, reagents containing two or more succinimidyl groups are small molecular weight crosslinkers including disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogues and derivatives. The above-referenced polymers are commercially available from Pierce (Rockford, Ill.). Another example of a small molecular weight crosslinker is penthaerythritol tetrasuccinimidyl glutarate (NHS-PET).

The term “synthetic” to refer to various polymers herein is intended to mean “chemically synthesized.” Therefore, a synthetic polymer in the present composition may have a molecular structure that is identical to a naturally occurring polymer, but the polymer per se, as incorporated in the composition of the invention, has been chemically synthesized in the laboratory or industrially. “Synthetic” polymers also include semi-synthetic polymers, i.e., naturally occurring polymers, obtained from a natural source, that have been chemically modified in some way. Generally, however, the synthetic polymers herein are purely synthetic, i.e., they are neither semi-synthetic nor have a structure that is identical to that of a naturally occurring polymer.

The term “synthetic hydrophilic polymer” as used herein refers to a synthetic polymer composed of molecular segments that render the polymer as a whole “hydrophilic,” as defined above. Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions. Hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol; polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines including poly(methyloxazoline) and poly(ethyloxazoline); and polyvinylamines.

Hydrophobic polymers can also be used to prepare the implant of the invention. Generally, “hydrophobic polymers” herein contain a relatively small proportion of oxygen and/or nitrogen atoms.

The term “aerosol” refers to a gaseous suspension of fine solid or liquid particles. The “propellant” is the driving force behind the aerosol. Liquefied propellants are gases that exist as liquids under pressure. Because the aerosol is under pressure the propellant exists mainly as a liquid, but it will also be in the head space as a gas. When the valve is opened, some of the liquid propellant turns to gas and keeps the head space full of gas. In this way the pressure in the can remains essentially constant and the spray performance is maintained throughout the life of the aerosol. The propellant is an essential element in the formulation. Compressed gas propellants really only occupy the head space above the liquid in the can. When the aerosol valve is opened the gas ‘pushes’ the biomaterial, in the form of a liquid, emulsion, or suspension out of the can. The amount of gas in the headspace remains the same but it has more space, and as a result the pressure will drop during the life of the can. Spray performance is maintained however by careful choice of the aerosol valve and actuator.

When referring to an active agent that may be incorporated into a composition, system, or bioimplant of the invention, it is to be understood that the term “active agent” encompasses not only any specified molecular entity but also its pharmaceutically acceptable, pharmacologically active analogs, including, but not limited to, salts, esters, amides, prodrugs, conjugates, active metabolites, and other such derivatives, analogs, and related compounds. By the terms “effective amount” and “therapeutically effective amount” of an active agent is meant a sufficient amount of the agent to provide the desired effect.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

As used herein, the terms “bioadhesive”, “biological adhesive”, and “surgical adhesive” are used interchangeably to refer to biocompatible compositions capable of effecting temporary or permanent attachment between the surfaces of two native tissues, or between a native tissue surface and either a non-native tissue surface or a surface of a synthetic implant.

The term “reactive moieties at the tissue site” refers to nucleophilic groups such as primary and secondary amines, sulfhydryl groups, hydroxyls and other chemical groups found in physiological fluids and on the tissue surface. The reactive moieties available to react with reactive groups of the implant can be found on amino acids, peptides, proteins, lipids, proteoglycans, cell surface proteins, extra cellular matrix, cell breakdown components, blood proteins and cells, wound exudates, blood, plasma and lymph.

The term “particle surface” refers to the total surface including the outer surface and surface created by porosity, texture of the surface, cracks, channels, or other structures available for contact with the components of physiological tissue fluids or tissue surface. In one aspect of the invention the particle surface is increased by grafting branched hydrophilic polymers with side chains containing multiple reactive groups.

The compositions and systems of the invention include at least one component that has reactive groups, i.e., “tissue-reactive” groups that react with functional groups on a tissue surface and/or with functional groups on one or more components of physiological fluid(s) present in the region of the tissue site to which the composition or system is applied. The reactive groups may be groups that can be further modified or activated to render said groups reactive. Among such groups are carboxylic acid groups. Since a carboxylic acid group per se is not susceptible to reaction with a nucleophilic amine or a sulfhydryl moiety, components containing carboxylic acid groups must be activated so as to be amine- or sulfhydryl-reactive. For example, a carboxylic acid can be reacted with an alkoxy-substituted N-hydroxy-succinimide or N-hydroxysulfosuccinimide in the presence of N,N-dicyclohexylcarbodimide (DCC) to form the reactive electrophilic groups N-hydroxysuccinimide ester and N-hydroxysulfosuccinimide ester, respectively. Carboxylic acids may also be activated by reaction with an acyl halide such as acyl chloride (e.g., acetyl chloride), to provide a reactive anhydride group. In a further example, a carboxylic acid may be converted to an acid chloride group using a thionyl chloride or an acyl chloride capable of an exchange reaction. Specific reagents and procedures used to carry out such activation reactions will be known to those of ordinary skill in the art and are described in pertinent texts and literature.

Activated or inherently reactive electrophilic groups can then react with the endogenous nucleophilic groups present at the tissue site where the composition or system is implanted. When the nucleophilic groups on the tissue surface are sulfhydryl groups, the reactive electrophilic groups include those that form thioester linkages upon reaction with the sulfhydryl group. Such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, for example 1-ethyl-3-[3-dimethylaminopropyl]carbodiimide can be used to facilitate the coupling of sulfhydryl groups to carboxyl-containing groups.

In addition to the sulfhydryl reactive groups that form thioester linkages, various other sulfhydryl reactive functionalities can be utilized to form other types of linkages. For example, compounds that contain methyl imidate derivatives form imido-thioester linkages with sulfhydryl groups.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones. This class of sulfhydryl reactive groups is particularly preferred because the thioether bonds may provide faster crosslinking and longer in vivo stability.

When higher molecular weight components are to be used in the compositions and systems of the invention, it is preferred that they have biodegradable linkages as described above, so that fragments larger than 15,000 mol. wt. are not generated during resorption in the body. In addition, to promote water miscibility and/or solubility, it may be desirable to add sufficient electric charge or hydrophilicity. Hydrophilic groups can generally be readily introduced using straightforward chemical syntheses, as long as they do not give rise to unwanted swelling or a disadvantageous decrease in compressive strength. In particular, polyalkoxy segments may weaken gel strength.

Synthetic hydrophilic polymers that are useful for the preparation of the biomaterial compositions and implantable systems of the invention include, but are not limited to: polyalkylene oxides, particularly polyethylene glycol and poly(ethylene oxide)-poly(propylene oxide) copolymers, including block and random copolymers; polyols such as glycerol; polyglycerol (particularly highly branched polyglycerol); propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol; polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like. Biodegradation, in the present context, is synonymous with “resorption” vis-à-vis the desired gradual degradation of the biomaterial compositions and systems of the invention following implantation into the body.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

Naturally occurring hydrophilic polymers useful as components of reactive groups herein include, but are not limited to: proteins such as collagen, fibronectin, vitronectin, laminin, elastin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans or their fragments and derivatives are preferred naturally occurring hydrophilic polymers for use herein.

Although a variety of different hydrophilic polymers can be used to prepare the compositions and systems of the invention, as indicated above, two of the preferred hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for preparation of reactive particles is a polyethylene glycol (PEG) having a molecular weight within the range of about 5 to about 15,000 mol. wt., although for a highly branched PEG, far higher molecular weight polymers can be employed—up to 1,000,000 or more—provided that biodegradable sites are incorporated ensuring that all degradation products will have a molecular weight of less than about 15,000. For most PEGs, however, the preferred molecular weight is about 3,000 to about 20,000 mol. wt., more preferably within the range of about 7,500 to about 15,000 mol. wt. Most preferably, the polyethylene glycol has a molecular weight of approximately 10,000 mol. wt.

Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Ala. (1997-1998). Activated PEG-containing compositions and systems of the invention may be prepared in various ways. In one of the approaches a commercially available PEG polymer having a pentaerythritol (2,2-bis(hydroxymethyl)-1,3-propanediol) core and a molecular weight of approximately 10,000 Da (prepolymer) can be crosslinked into a three-dimensional polymer by reaction with glutaryl dichloride in the presence of pyridine as a base. The crosslinked PEG can be formed into an implant of a desired shape or into particles of different sizes by mechanical grinding after the crosslinking step or by conducting the crosslinking process in a micellar system where a prepolymer is crosslinked in micelles of the desired size. The crosslinking process conditions can be optimized such that the formed particles contain carboxyl groups in the form of glutaryl groups. Alternatively, additional carboxyl groups can be readily prepared by conversion of the exposed, unreacted hydroxyl groups to carboxylic acid groups using a reaction with an anhydride in the presence of a nitrogenous base. The carboxyl groups on the particles can be activated by esterification with N-hydroxysuccinimide, N-hydroxysulfosuccinimide, or the like, to prepare polyfunctionally activated PEG implants or particles. Additional forms of activated particles are functionally activated PEG glycidyl ether particles, PEG-isocyanate particles, and PEG-vinylsulfone particles.

The biomaterial compositions and implantable systems of the invention can also be prepared from hydrophobic polymers, particularly when more gradual resorption is desired. Polylactic acid and polyglycolic acid are examples of two hydrophobic polymers that can be used.

Many low molecular weight, multifunctional crosslinkers can be used in the preparation of reactive particles, for example in the crosslinking of prepolymers into particles or as particle surface activating groups. A free multifunctional crosslink may be formed with either reactive or inert particles such that the crosslinker is able to diffuse from the particles into the surrounding tissue prior to reacting with tissue moieties. Such multifunctional free crosslinkers may be useful for the further crosslinking of moieties present at the tissue site because they will be able to diffuse into the surrounding tissues after an initial bioimplant is formed. The diffusion of these small crosslinkers is particularly beneficial because further mixing of reactive particles with tissue moieties can sometimes be constrained by the gel-like or solid nature of the bioimplant.

As indicated above, the molecular core of one or more free crosslinkers can also be a low molecular weight compound, i.e., a C₂-C₁₄ hydrocarbyl group containing zero to 2 heteroatoms selected from N, O, S and combinations thereof. Such a molecular core can be substituted with electrophilic groups. One preferred low molecular weight crosslinker is a pentaerythritol tetra-succinimidyl glutarate (NHS-PET). Free high molecular weight crosslinkers can also be used but they will be less effective due to their limited diffusion potential.

Low molecular weight di- and poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives. The aforementioned compounds are commercially available from Pierce (Rockford, Ill.). Such di- and poly-electrophiles can also be synthesized from di- and polyacids, for example by reaction with an appropriate molar amount of N-hydroxysuccinimide in the presence of DCC.

The invention is also directed to a biomaterial composition and implantable system comprised of two or more types of reactive particles, where the first particle type contains electrophilic groups and the second particle type contains nucleophilic groups reactive with the electrophilic groups on the first particle type. Examples are N-succinimide ester activated particles and amine- or sulfhydryl-containing particles. This type of particle-particle crosslinking system is particularly effective when said particles are relatively small, present at a relatively high concentration and are mixed prior to the onset of crosslinking. In one embodiment of such invention, a dry mixture of small insoluble electrophilic (for example NHS-activated particles) and nucleophilic PEG particles (for example sulfhydryl carrying particles) ranging in size from about 10 μm to 100 μm can be applied onto a surgical tissue site. Upon rehydration the particle mixture becomes covalently crosslinked. Optionally the dry mixture of particles also contains buffer salts that upon dissolution by physiological fluids at the tissue site create an elevated pH which increases the rate of the particle crosslinking reaction.

The particles carrying electrophilic groups are able to react with both nucleophile-carrying particles and with nucleophilic moieties present at the tissue site and on the tissue surface. The resultant bioimplant is composed of particles, components of physiological fluids which have been crosslinked or physically entrapped, and crosslinks with the tissue surface.

Biomaterial compositions and implantable systems according to the present invention can be prepared from synthetic polymers, natural polymers, hydrophilic polymers, hydrophobic polymers, organic salts, inorganic salts, ceramics, hydroxyapatite, tricalcium phosphate, sol gels, organosilanes, sea coral, demineralized bone, glass, metal oxides (e.g. TiO₂), metals, lipids, polysaccharides, gold, silver, titanium, talc, Teflon, e-PTFE, Dacron, carbon, hydrogels, elastomers, plastics, metal alloys, cellulose, oxidized cellulose, hyaluronic acid, polymers of drugs, silicone, or combinations of thereof.

For instance, stabilized magnetic ferro-colloids or magnetic fluids can be used as a part of a biomaterial composition or implantable system of the invention. A magnetic fluid can be prepared as a stable colloid containing dextran and iron-oxide. Such magnetic fluids are typically composed of particles of magnetite coated with dextran. A reactive group containing a dextran derivative can be easily prepared by acylating commercially available amino dextran. Once prepared, the carboxyl-dextran on the surface of the particles can be activated using NHS and carbodiimide reagents into an NHS-ester. Magnetic fluids and particles with reactive groups can be used for multiple uses including immunodiagnostic reagents, cell labeling and magnetic separation, local concentration of magnetic particles by applying a gradient of magnetic field to the target site in the living body, and for coating magnetic particles with plasma proteins to increase time of their circulation in the bloodstream.

Gold particles and colloids can also be used. For example, commercially available gold particles ranging in size between 1 nm and 1000 nm can be coated using polymers or small molecules containing free carboxyl and thiol groups. Thiol groups strongly bind to the surface of gold. The carboxyl groups on the surface of the gold particles then can be modified into NHS-esters. One example of a small molecule containing both carboxylic and sulfhydryl groups is dimercaptosuccinic acid (DMSA). DMSA can be used to coat the surface of gold. The carboxyl groups can potentially be activated by converting them into N-hydroxysuccinimide esters after DMSA is immobilized on the gold surface. To increase the reactivity of the activated surface groups, a hydrophilic linking group or polymer can be covalently linked to the carboxyl groups of DMSA on the gold surface to provide a spacer. The terminal group of the spacer can be modified to provide a nucleophilic or electrophilic group in various ways, as will be appreciated by those of ordinary skill in the art.

The presence of reactive groups on the surface of the bioimplant is beneficial for long term tissue compatibility. For example, when contacted by physiological fluids, the reactive groups react with the proteins in the fluids, effectively covering the bioimplant with an autologous protein coating that is covalently bound to the implant surface. This coating can be particularly useful when microparticles are used as drug delivery vehicles in the implantable system, so that drug release into the systemic circulation is prolonged. Coating particles with autologous proteins increases their circulation time by preventing the particles from being retained by reticulo-endothelial systems in the liver, lungs, kidneys and spleen.

Particles, wound closure and tissue repair devices can be prepared by covalently crosslinking monomers or prepolymers, by condensation from reactive prepolymers, by charge interaction, by hydrophobic interaction, by physical crosslinking, by temperature precipitation, by solvent precipitation, by co-precipitation with other component, by drying, by freeze-drying, by phase separation, by emulsification, by sonication, by extrusion, by spray-drying, by denaturation and by many other methods known in the art.

Activated compositions and systems of the invention can be prepared by reacting a water soluble polymer such as pentaerythritol polyethylene glycol ether with a molecular weight of approximately 10,000 (see, e.g., U.S. Pat. No. 6,458,889), with a crosslinking reagent to form a water insoluble matrix that can be processed into particles of the desired size. Since pentaerythritol polyethylene glycol ether has four end terminal hydroxyl groups that can participate in the formation of a tri-dimensional matrix and only three reactive groups are sufficient to form a tri-dimensional matrix during crosslinking, some hydroxyl groups or their derivatives (ibid.) are not crosslinked and can be used to form reactive groups. The level of matrix crosslinking and number of free groups can be controlled by the molar proportion of pentaerythritol polyethylene glycol ether and crosslinker and by controlling the conditions of the matrix formation reaction.

An implant or bioimplant prepared from the compositions and systems of the invention can have a shape designed to be useful for various surgical procedures. For example, it can be shaped in the form of a tube, a screw, a coil, a blade, an arrow, a hook, a spike, a needle, a rod, a cylinder, a sphere, any other geometrical or custom designed shape, or have the shape of an existing medical device. The implant can be used for the repair of blood vessels as an extravascular or intravascular connector shaped in the form of a tube, or for nerve repair as a tube-shaped connector placed over a nerve. Similarly, a tube-shaped connector can be used for the repair of the intestines, esophagus, urethra, ureter, fallopian tubes, or broncho-alveolar tree as an internal or external connector. When shaped in a form designed to penetrate and anchor into the tissues, said medical device can be used for wound closure, or the repair of parenchymal tissues, cartilage, ligaments, bone, tendon, muscle, skin, liver tissue, lung tissue, stomach tissue, intestines, uterus, bladder, kidneys, eye tissues, spleen, heart muscle, heart blood vessels, urethra, fallopian tubes, central nervous tissue, blood vessels, peripheral nerves, or ureters. When the implant has a lumen filling shape or has the shape of a sphere, cylinder or rod, or is a suspension of particles, said implant can be used for repair of cerebral vascular aneurisms, or as a vascular embolic agent, or as a filler of bone and cartilage defects.

The methods and compositions of the invention can also be implemented to provide a medical device having a coating of reactive groups. Such a coating can be used to effectively immobilize the device at a desired anatomical location without the use of surgical sutures, staples, or adhesives. Such medical devices include, but are not limited to, implantable sensors, pacemakers, implantable defibrillators, implantable pumps, implantable electrostimulators, implantable RFID devices, immunoisolation devices with insulin-producing cells used for the treatment of diabetes, prosthetic meshes, implantable orthopedic devices, and indwelling catheters.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains.

### EXPERIMENTAL

The practice of the present invention will employ, unless otherwise indicated, conventional techniques of organic chemistry, polymer chemistry, and pharmaceutical formulation, which are within the skill of the art. Such techniques are explained fully in the literature.

### EXAMPLE 1

### Synthesis of Reactive PEG Particles

Pentaerythritol polyethylene glycol ether (schematically shown in FIG. 1) with molecular weight of approximately 10,000 g/mol is dissolved in anhydrous dimethylformamide (DMF) to form a 2% (w/w) solution.

Half of the volume of this solution is added drop-wise to an anhydrous DMF solution containing 1.8 mole equivalents of glutaryl dichloride and 4 mole equivalents of pyridine, with stirring, to form the reaction mixture. The reaction mixture is incubated for 1 hour at 40° C. and then chilled to 4° C. The second half of the original solution is chilled to 4° C., and then quickly added, with vigorous mixing, to the reaction mixture. Quickly after this addition, 4 volumes excess of hexane that is chilled to 4° C. is added to the reaction mixture. The resulting mixture is emulsified using agitation and/or a sonicator bath. After a stable emulsion condition is achieved, the temperature of the reactive mixture is increased to 40° C. and the reaction is continued for 20 hours. The resulting particles are washed with DMF. Five mole equivalents (relative to the initial PEG prepolymer amount) of N-hydroxysuccinimide and 5 mole equivalents of N,N′-dicyclohexyl-carbodiimide (DCC) in anhydrous DMF are added, and the mixture is incubated for 18 h at 40° C. The particles are washed using an appropriate anhydrous solvent to remove dicyclohexylurea, and then washed with anhydrous tetrahydrofuran (THF) at 40° C. The particles are dried under dry nitrogen.

### EXAMPLE 2

### Sprayable Hemostatic Sealant

Dry particles from Example 1 are rehydrated in 150 mM NaCl and 20 mM sodium phosphate buffer at pH 6.0.

A 40% by volume suspension of rehydrated PEG particles in 130 mM NaCl 100 mM sodium phosphate buffer is prepared at pH 8.5. Immediately after preparing the activated PEG particle suspension, the particle suspension is applied to the tissue surface with diffuse bleeding in the form of spray or stream such that the applied formulation mixes effectively with the physiological fluid present at the tissue site and is in contact with the tissue surface. One to 5 minutes is allowed for the PEG particles to react with tissue surfaces and components of the physiological fluids present at the tissue site. Hemostatic sealing and hemostasis at the bleeding site is observed.

### EXAMPLE 3

### Hemostatic Sealant Aerosol

Reactive particles from Example 1 are sieve dried through a 50 micron sieve mesh. 1 g of dry reactive PEG particles less than 50 micron in size are blended with the finely ground dry residue (containing particles of less then 5 microns in size) that remains after drying 20 ml of 0.3 M sodium carbonate buffer at pH 9.5. A 25% suspension of this blend in anhydrous ethanol is prepared. Using a Preval® Sprayer ( Precision Valve Corporation, Yonkers, N.Y.), this suspension is applied onto a diffusely bleeding tissue site while holding the sprayer 1-5 inches from the tissue surface. Hemostatic sealing at the tissue site is achieved and observed after 1-3 minutes.

### EXAMPLE 4

### Hemostatic Sealant Aerosol

A blend of dry PEG particles and buffer salts is prepared as described in Example 3. The blend of dry PEG particles and buffer salts is formulated with an aerosol propellant composed of 1,1,1,2-tetrafluoroethane or fluorotrichloromethane/dichlorodifluoromethane (at a 30/70% proportion). The formulation is package in the liquefied form into a sealed aerosol can commonly used for the delivery of powder form pharmaceutical products. The aerosol of PEG particles and buffer salts is applied onto a diffusely bleeding tissue site 1 to 5 inches from the tissue surface. Hemostatic sealing at the tissue site is observed after 1-3 min.

### EXAMPLE 5

### Reactive Sealant for Surgical Adhesion Prevention

The aerosol from Example 4 is applied on a surgical suture line and surrounding tissues in myomectomy surgery to uniformly seal the tissue surface. A reduction in the prevalence and severity of adhesions at the aerosol treated site vs. untreated controls is observed during laparoscopic observation of the treated site 6-12 weeks after surgery.

### EXAMPLE 6

### Biomaterial for Vascular Aneurysm Repair

A 50% suspension of the blended PEG particles and buffer salts, as in Example 3, is prepared in dimethylsulfoxide (DMSO) or dimethylformamide (DMF) solvents. This suspension is delivered to a cerebral aneurism using a catheter. The particles are allowed to rehydrate, the buffer salts are allowed to dissolve, and present blood is allowed to admix with particles to form a bioimplant which fills the aneurysm.

### EXAMPLE 7

### Hemostatic Sealant for Tissue Biopsy Canal Repair

The suspension from Example 6 is delivered to a tissue biopsy canal (lung, liver, spleen, brain, tumor, bone, etc.). The particles are allowed to rehydrate, the buffer salts are allowed to dissolve, and delivered formulation is allowed to form a bioimplant which fills the biopsy canal.

### EXAMPLE 8

### Hemostatic Sealant for PTCA Procedure

The suspension from Example 6 is delivered into the percutaneous access space at the end of a PTCA procedure. The particles are allowed to rehydrate, the buffer salts are allowed to dissolve, and delivered formulation is allowed to form a bioimplant which seals PTCA access and prevents femoral artery bleeding.

### EXAMPLE 9

### Hemostatic Sealant for Laparoscopic Surgery

The aerosol from Example 4 is applied onto a bleeding site using a laparoscopic delivery nozzle compatible with laparoscopic access ports. Hemostatic sealing of the bleeding site is observed.

### EXAMPLE 10

### Cartilage Repair Biomaterial with Reactive Groups

The suspension from Example 6 is delivered to the site of a cartilage defect using an arthroscopic delivery system. The delivered formulation is allowed to rehydrate, react with synovial fluid components and form a bioimplant attached to the surrounding cartilage tissue.

### EXAMPLE 11

### Adhesive Spray for Hemostasis and Wound Closure

The aerosol of blended PEG particles and buffer salts from Example 3 is applied onto opposing tissue surfaces in a surgical wound to uniformly cover approximately 50% each of opposing tissue surfaces with the PEG particles. The treated, opposing tissue surfaces are contacted together, with contact between the tissue surfaces being maintained for 2 minutes. Wound closure strength is observed by testing the force required to cause dehiscence of the wound after 20 minutes.

### EXAMPLE 12

### Bone Repair Biomaterial with Reactive Groups

A 2 ml blend of PEG reactive particles and buffer salts (corresponding to 4 ml of buffer) is prepared as in Example 3, using 2 ml hydroxyapatite/tricalcium phosphate (HA/TCP) commonly used for bone repair. The dry blend is placed or packed into a bone defect containing physiological fluids. If bleeding is present, hemostatic sealing is observed. 8-12 weeks after implantation, healing and bone regeneration at the implant site is observed by X-ray imaging.

### EXAMPLE 12

### Bone Repair Biomaterial with Small Molecular Weight Crosslinker

HA/TCP particles commonly used as a component of bone cement formulations and for ceramic coatings of orthopedic devices are obtained. A blend of 5 ml HA/TCP particles and buffer/salts corresponding to a 5 ml buffer is prepared as in Example 3. Pentaerythritol (available from Sigma Aldrich) is reacted with a 25 molar excess of glutaric anhydride in the presence of a 40 mole excess of pyridine. The ester of glutaric acid and pentaerythritol is then purified by gel permeation chromatography, and an N-hydroxysuccinimide ester derivative is prepared by reacting this ester with a 20 molar excess of N-hydroxysuccinimide (NHS) and a 20 molar excess of N,N′-dicyclohexyl-carbodiimide (DCC). 4-(succinimidyl-glutarate)pentaerythritol (NHS-PET, shown in FIG. 3) is then isolated by gel permeation chromatography or by organic solvent extraction. The HA/TCP particles and buffer/salts blend previously prepared is soaked in, or impregnated with, a solution of NHS-PET in anhydrous organic solvent. The resulting particles are then dried under anhydrous conditions. These dry particles are used to repair bone defects by packing particles into the defect in the presence of physiological fluids (e.g., serum, blood, lymph, wound exudate). The NHS-PET is allowed to dissolve in tissue fluids and crosslink components of the physiological fluids are allowed to form a hemostatic bioimplant. Bone healing is observed by X-ray at the implantation site after 8-12 weeks following the surgery.

### EXAMPLE 13

### Adhesive Tape for Surgical Wound Closure

A tape, patch, or mesh is prepared from a biocompatible, bioresorbable material (implant) which has chemical moieties suitable for modification and activation. Such implant material can be prepared by polymerization or crosslinking of prepolymers and can include PEG, methacrylate, polyvinyl alcohol and other polymer backbones or their block co-polymers containing side chain groups that can be activated. As a specific example, a sheet of material is formed by crosslinking PEG prepolymer as in example 1. Holes or openings of 2-4 mm in diameter are formed in the PEG sheet such that the total surface area of holes or openings corresponds to approximately 30% of the total tape or patch surface. The carboxyl groups on the PEG implant are activated using N-hydroxysuccinimide and DCC reagents. A buffer salt residue, as described in Example 3, is deposited on the implant in an appropriate amount corresponding to the size of the implant and anticipated size of the tissue site. Optionally, the activated PEG implant is impregnated with a small molecular weight crosslinker such as NHS-PET, as described in Example 12, and the implant is dried. The implant is placed between opposing surfaces of a surgical wound. The opposing surfaces are contacted such that the implant is in contact with both sides and the opposing tissues contact each other through the openings in the implant. 2-5 minutes is allowed for the implant to rehydrate and react with tissue surfaces and components of physiological fluids. The wound closure strength is observed by testing the force required to cause dehiscence of the wound.

### EXAMPLE 14

### Sutureless Wound Closure Device

An implant for sutureless wound closure is prepared from a dehydrated biocompatible polymer formed to penetrate soft tissues and anchor inside of the tissue. The implant has at least two ends shaped to facilitate easy tissue penetration and anchorage (i.e. double-sided arrow with hooks that allows easy insertion into the tissue but prevents ready removal of the device). The implant is capable of swelling 20% to 400% upon rehydration. Optionally, the implant contains in its structure activated groups such as NHS esters of carboxyl groups on the surface of the implant. To provide an environment for faster reactions between the NHS-activated groups on the implant with reactive groups on the tissue surfaces and in the physiological fluids (i.e. —NH₂ and —SH), an appropriate amount of buffer salts is formulated with the implant similar to Example 3. Further and also optionally, the implant contains a small molecular weight crosslinker capable of reacting with components of physiological fluids present at the wound site and tissue surfaces (for example NHS-PET as described in Example 12) upon dissolution in tissue fluids. One end of the device is inserted in the tissue surface to be surgically closed. The second end of the device is inserted into the opposing tissue surface such that both tissue sides are in contact with each other in a surgically desirable manner. The implant is allowed to rehydrate and anchor by hydrostatic swelling, and activated groups are allowed to react with the tissue moieties at the tissue site for 5-20 minutes. The strength of wound closure is tested by measuring a wound dehiscence force.

### EXAMPLE 15

### An Activated Prosthetic Device and Drug Delivery Vehicle

Reactive groups on the surface of a medical device are prepared using a biocompatible polymeric material such as hernia repair meshes, nasal tubes, ear tubes, pins, clips, vascular prostheses, anastomic stents, intravascular stents, extravascular stents, vascular bypass prosthesis, embolic particles and beads, film, tape, rods, fibers, sutures, drainage tubes, catheters, hip implants, etc. For example, the device is prepared by modifying hydroxyl or carboxyl groups on the surface of the medical device (i.e. NHS-, or maleimide-derivatives). Optionally, a prosthetic device contains a buffer and salt residue on the surface of the device to provide for an alkaline environment that speeds up reaction between reactive groups on the device with amino and sulfhydryl groups present at the tissue site. Further and also optionally, the medical device may contain a small molecular weight crosslinker that is not covalently bound to the device's polymeric structure and is not capable of reacting with the groups present on the device. Further and also optionally, the medical device may contain a drug described herein. The device is surgically placed at a target location in the body and is immobilized by being held in position for 1-10 min. Crosslinking by the reactive groups on the device surface and by an optionally present, small molecular weight crosslinker that diffuses and reacts with the surrounding tissues provides for device immobilization at the target site without the use of sutures, staples or separately added surgical adhesives. Any means of surgical immobilization of the device in the target location are released, and the strength of device adherence is tested by a method appropriate to the application. Clinical outcomes and benefits of using sutureless wound closure as described in this example are evaluated and compared with conventional techniques using sutures and staples.

### EXAMPLE 16

### Crosslinking and Delivery of Autologous Tissue Preparations

The reactive PEG particle formulated with the propellant as described in Example 4 are placed in an aerosol can similar in design to a Preval® Sprayer ( Precision Valve Corporation, Yonkers, N.Y.), where an external formulation (e.g., paint) can be dispensed together with the propellant and the reactive particles formulated with the propellant (see FIG. 8). A preparation of autologous platelets, cartilage cells, mesenchimal cells, stem cells, or any other cells is placed in the Preval® Sprayer container (or similar device) for delivering an external formulation (e.g., paint). The autologous tissue preparation is applied onto the target tissue site while holding the sprayer 1-5 inches from the tissue surface. Accumulation and immobilization of the autologous tissue preparation crosslinked by the reactive PEG particles is observed at the target tissue site in less then 1 minute.

1. A biomaterial composition comprising particles comprised of a biocompatible polymer, the particles having tissue-reactive groups that react with endogenous nucleophilic groups present at a tissue site in a mammalian body to form new covalent bonds, ionic bonds, or both covalent bonds and ionic bonds, wherein the particles are in a particle population having a median diameter in the range of about 1 nm to about 3 mm.

2. The composition of claim 1, comprising a resorption rate-controlling component having an aqueous solubility selected such that the composition is resorbed after introduction into a living mammalian body over a time period of not less than 3 hours.

3. The composition of claim 2, wherein the median diameter is in the range of about 1 μm to about 100 μm.

4. The composition of claim 3, wherein the median diameter is in the range of about 25 μm to about 75 μm.

5. The composition of claim 2, wherein the biocompatible polymer is substituted with the tissue-reactive groups.

6. The composition of claim 5, wherein the biocompatible polymer is a hydrophilic, water-swellable polymer.

7. The composition of claim 2, wherein the resorption rate-controlling component is the biocompatible polymer.

8. The composition of claim 2, further comprising an additional biocompatible polymer.

9. The composition of claim 8, wherein the additional biocompatible polymer is substituted with the tissue-reactive groups.

10. The composition of claim 9, wherein the additional biocompatible polymer is a hydrophilic, water-swellable polymer.

11. The composition of claim 8, wherein the additional biocompatible polymer is the resorption rate-controlling component.

12. A biomaterial composition comprising a population of porous particles having a median diameter in the range of about 1 nm to about 3 mm and a biocompatible polymer associated with the particles but not covalently bound thereto, the particles having tissue-reactive groups that react with endogenous nucleophilic groups present at a tissue site in a mammalian body to form new covalent bonds, ionic bonds, or both covalent bonds and ionic bonds.

13. The composition of claim 12, wherein the porous particles comprise a nonpolymeric resorption rate-controlling component having an aqueous solubility selected such that the composition is resorbed after introduction into a living mammalian body over a time period of not less than 3 hours.

14. The composition of claim 13, wherein the median diameter is in the range of about 1 μm to about 100 μm.

15. The composition of claim 14, wherein the median diameter is in the range of about 25 μm to about 75 μm.

16. The composition of claim 13, wherein the biocompatible polymer is substituted with the tissue-reactive groups.

17. The composition of claim 16, wherein the biocompatible polymer is a hydrophilic, water-swellable polymer.

18. The composition of claim 13, further comprising an additional biocompatible polymer.

19. The composition of claim 18, wherein the additional biocompatible polymer is substituted with the tissue-reactive groups.

20. The composition of claim 19, wherein the additional biocompatible polymer is a hydrophilic, water-swellable polymer.

21. The composition of claim 13 or claim 17, wherein the porous particles are impregnated with the biocompatible polymer.

22. The composition of claim 13 or claim 17, wherein the porous particles are coated with the biocompatible polymer.

23. The composition of claim 13 or claim 17, wherein the tissue-reactive groups are on the surface of the porous particles.

24. The composition of claim 1 or claim 12, wherein the tissue-reactive groups are electrophilic groups.

25. The composition of claim 24, wherein the electrophilic groups are selected from alkenyloxycarbonyl, acyl, acyloxy, chlorocarbonyl, formyl, halo, halocarbonyl, isocyano, isothiocyano, alkenyl, and epoxy.

26. The composition of claim 25, wherein the electrophilic groups are alkenyloxycarbonyl groups.

27. The composition of claim 24, wherein the electrophilic groups are activated ester groups.

28. The composition of claim 27, wherein the electrophilic groups are selected from acrylate and methacrylate groups.

29. The composition of claim 1, wherein the particles are prepared by crosslinking polyethylene glycol and the tissue-reactive groups are N-hydroxysuccinimide esters of carboxylic acid groups.

30. The composition of claim 1 or claim 12, further comprising an effective amount of an active agent.

31. The composition of claim 30, wherein the active agent is a therapeutically active agent.

32. The composition of claim 1 or claim 12, in substantially anhydrous form.

33. The composition of claim 13, wherein the nonpolymeric resorption rate-controlling component is selected from metals, metal alloys, organometallic compounds, inorganic gels, salts, hydrophobic organic compounds, and ceramics.

34. The composition of claim 33, wherein the nonpolymeric resorption rate-controlling component is selected from metals, ceramics, and salts.

35. The composition of claim 34, wherein the nonpolymeric resorption rate-controlling component is selected from gold particles, silica particles, hydroxyapatite, tricalcium phosphate, titanium oxide, and lipids.

36. A biocompatible, implantable system for implantation into a mammalian body at a tissue site to form a bioimplant, comprising: (a) a biomaterial composition having tissue-reactive groups selected from electrophilic groups and nucleophilic groups that react with endogenous groups present at a tissue site in a mammalian body to form new covalent bonds, ionic bonds, or both covalent bonds and ionic bonds, wherein, if the biomaterial composition has both electrophilic groups and nucleophilic groups, then under predetermined conditions either the electrophilic groups or the nucleophilic groups, but not both, react with the endogenous groups; and (b) a resorption rate-controlling component having an aqueous solubility selected such that the composition is resorbed after introduction into a living mammalian body over a time period of not less than 3 hours.

37. A biocompatible, implantable system for implantation into a mammalian body at a tissue site to form a bioimplant, the composition comprising: (a) porous particles having a median diameter in the range of about 1 μm to about 100 μm and comprising a material selected such that the bioimplant is resorbed over a time period of not less than 3 hours; and (b) impregnated therein, a biocompatible polymer having tissue-reactive electrophilic groups that react with endogenous nucleophilic groups present at the tissue site to form new covalent bonds, ionic bonds, or both covalent bonds and ionic bonds.

38. The system of claim 36 or claim 37, in substantially anhydrous form.

39. The system of claim 36 or claim 37, further comprising a buffer.

40. The system of claim 36 or claim 37, further including an effective amount of an active agent.

41. The system of claim 40, wherein the active agent is a therapeutically active agent.

42. The system of claim 36 or claim 37, further comprising a nonpolymeric material capable of polymerization upon contact with the tissue surface.

43. The system of claim 42, further comprising a polymerization initiator.

44. The system of claim 36 or claim 37, further comprising a visualization component.

45. The system of claim 36 or claim 37, further comprising at least one additional component selected from living cells, stem cells, autologous cells, cell fragments, non-living cells, viruses, plasmids, prions, and bacteria.

46. A bioimplant comprising a biocompatible composition covalently bound to a surface of a tissue in a living mammalian body and to components of endogenous physiological fluids present in the region of the tissue, wherein the composition is comprised of porous particles having a median diameter in the range of about 1 μm to about 100 μm and comprised of a material selected such that the bioimplant is resorbed over a time period of not less than 3 hours, and a biocompatible polymer that provides the covalent binding by virtue of having tissue-reactive groups that react with endogenous nucleophilic groups.

47. A bioimplant formed by introducing a predetermined quantity of the system of claim 36 or claim 37 into a living mammalian body at a tissue site.

48. The bioimplant of claim 46, wherein the system is introduced by application onto a tissue surface.

49. A device for implanting a biocompatible composition into a living mammalian body at a predetermined tissue site, comprising the composition of claim 1 or claim 12 and a means for introducing the composition into the body at the predetermined tissue site so that a bioimplant is formed.

50. A device for implanting a system into a living mammalian body at a predetermined tissue site, comprising the implantable system of claim 36 or claim 37 and a means for introducing the system into the body at the predetermined tissue site so that a bioimplant is formed.

51. A propellant driven delivery system for delivery into a living mammalian body of an implantable biocompatible composition having tissue-reactive groups, comprising the implantable biocompatible composition, a propellant, and a device that contains the composition and the propellant and includes a means for ejecting the composition in aerosolized form.

52. The system of claim 51, wherein the propellant is housed in a first compartment of the device and the composition is housed in a second compartment of the device.

53. The system of claim 52, wherein the device further includes a nozzle in which the composition and the propellant are combined prior to ejection in aerosolized form.

54. A method for surgical repair of living tissue in a mammalian body, comprising introducing the composition of claim 1 or claim 12 into the body at a tissue site requiring joinder of adjacent but separate regions of tissue.

55. A method for surgical repair of living tissue in a mammalian body, comprising introducing the system of claim 36 or claim 37 into the body at a tissue site requiring joinder of adjacent but separate regions of tissue.

56. A method for the delivery of an active agent to a tissue site in a living mammalian body, comprising introducing the composition of claim 30 into the body at a tissue site where the active agent is to be deposited.

57. A method for the delivery of an active agent to a tissue site in a living mammalian body, comprising introducing the system of claim 40 into the body at a tissue site where the active agent is to be deposited.

58. A device for wound closure or surgical repair having a size and shape suitable for application to a selected tissue site in need of closure or repair and comprised of: (a) a biomaterial composition having tissue-reactive groups selected from electrophilic groups and nucleophilic groups that react with endogenous groups present at a tissue site in a mammalian body to form new covalent bonds, ionic bonds, or both covalent bonds and ionic bonds, wherein, if the biomaterial composition has both electrophilic groups and nucleophilic groups, then under predetermined conditions either the electrophilic groups or the nucleophilic groups, but not both, react with the endogenous groups; and (b) a resorption rate-controlling component having an aqueous solubility selected such that the composition is resorbed after introduction into a living mammalian body over a time period of not less than 3 hours.
